(19) United States
(12) Patent Application Publication
(10) Pub. No.: US 2022/0211695 A1
(43) Pub. Date: Jul. 7, 2022

(54) MORPHINE-INDUCED RESPIRATORY DEPRESSION: BEHAVIORAL, PHRENIC AND BRAINSTEM RESPIRATORY NEURONAL EVIDENCE

(71) Applicant: GEORGIA STATE UNIVERSITY RESEARCH FOUNDATION, INC., Atlanta, GA (US)

(72) Inventors: HAO XING, Atlanta, GA (US); CHUN JIANG, Atlanta, GA (US); NATHAN N. SABATE, Atlanta, GA (US); CHRISTOPHER M. JOHNSON, Atlanta, GA (US)

(21) Appl. No.: 17/612,152

(22) PCT Filed: May 18, 2020
(86) PCT No.: PCT/US2020/033360

§ 371 Date: Nov. 17, 2021

Related U.S. Application Data
(60) Provisional application No. 62/852,017, filed on May 23, 2019.

(51) Int. Cl. A61K 31/485; A61K 31/4453; A61P 11/00

(52) U.S. Cl. CPC A61K 31/485; A61P 11/00; A61K 31/4453

(57) ABSTRACT

Disclosed is a method for reducing opioid-induced breathing, phrenic and rVRG abnormal activities. The disclosed methods can maintain the desired effects of an opioid while reducing any unwanted breathing problems.

### FEDERALLY SPONSORED RESEARCH

This discovery was made by government support under grant number (R01-NS-073875) by National Institutes of Health (NIH). The government has certain rights in the invention.

### FIELD

Disclosed is a method for reducing opioid-induced breathing, phrenic and rVRG abnormal activities. The disclosed methods can maintain the desired effects of an opioid while reducing any unwanted breathing problems.

### BRIEF DESCRIPTION OF THE FIGURES

FIG. 1 depicts the experimental timeline wherein morphine is injected and cloperastine is injected after morphine injection at t=0.

FIG. 2 depicts the experimental timeline wherein cloperastine is injected and morphine is injected after cloperastine injection at t=0.

FIG. 3 A1 is a phethysmography recording of the baseline prior to infusion of active ingredients. A2 is a phrenic recording according to the breathing activity.

FIG. 4 A1 is a phethysmography recording 20 minutes after morphine injection. A2 is a phrenic recording 20 minutes after morphine injection.

FIG. 5 A1 is a phethysmography showing the maximum effect of morphine injection. A2 is a phrenic recording showing the maximum effect of morphine injection.

FIG. 6 depicts a breathing baseline for the experiment wherein morphine is first given followed by cloperastine.

FIG. 7 is the breathing after injection of 10 mg/kg morphine to the subject of FIG. 6.

FIG. 8 shows the reduction in erratic breathing after injection of 30 mg/kg of cloperastine.

FIG. 9 depicts a breathing baseline for the experiment wherein morphine is first given followed by cloperastine.

FIG. 10 is the breathing after injection of 30 mg/kg of cloperastine 10 mg/kg morphine to the subject of FIG. 9.

FIG. 11 shows the reduction in erratic breathing after injection of 10 mg/kg morphine. The breathing pattern is similar to that of FIG. 8.

FIG. 12 depicts the time course of the normalized effect on breathine amplitude. The solid red line shows the effect that an injection of cloperastine has on morphine breathing suppression (top black arrow).

FIG. 13 depicts the time course of the normalized effect on breathine frequency. The solid red line shows the effect that an injection of cloperastine has on reducing the effects of morphine of breathing frequency (top black arrow).

FIG. 14 is a normalized plot of the effect on breathing amplitude. The solid blue line represents cloperastine treatment following treatment by morphine. Dotted red line represents morphine treatment in the absence of cloperastine. Solid red line indicates morphine after cloperastine treatment.

FIG. 15 is a normalized plot of the effect on breathing frequency. The solid blue line represents cloperastine treatment at 20 minutes following treatment by morphine. Dotted red line represents morphine treatment in the absence of cloperastine. Solid red line indicates morphine after cloperastine treatment after 20 minutes.

FIG. 16 is the normalized effects of these experiments on minute ventilation. Cloperastine is given at 20 minutes post morphine and as indicated by the top arrow, a significant change occurs by about 45 minutes.

FIG. 17 is a plot of the phrenic discharge pattern of a control subject.

FIG. 18 is a plot of the phrenic discharge pattern of a subject after injection of 50 mg/kg morphine.

FIG. 19 is a plot of the phrenic discharge pattern of a subject after injection of 50 mg/kg morphine followed by an injection of 30 mg/kg of cloperastine. The erratic phrenic activity has nearly completely subsided.

FIG. 20 is a chart depicting the effects of morphine and morphine/cloperastine on inspiratory (Ti) and expiratory durations (Te) in experimental animals in microseconds. The morphine/cloperastine treatment returns Ti duration to near normal baseline.

FIG. 21 shows that the morphine/cloperastine treatment decreases the Ti/Te to less than half of the morphine value.

FIG. 22 compares the phrenic firing frequency between control, morphine only and the morphine/cloperastine treatment.

FIG. 23 compares the breath volumes between control, morphine only and the morphine/cloperastine treatment.

FIG. 24 depicts the effect of cloperastine treatment and morphine/cloperastine treatment versus control for both Te and Ti duration in milliseconds. The Te and Ti of cloperastine are nearly the same as the control.

FIG. 25 depicts the ratio of Te/Ti of the cloperastine treatment and morphine/cloperastine treatment versus control. The Te/Ti ratio for cloperastine is nearly identical to control.

FIG. 26 depicts the breathing frequency of the cloperastine treatment and morphine/cloperastine treatment versus control. The breathing frequency for cloperastine is nearly identical to control.

FIG. 27 compares the breath volumes between control, cloperastine and the morphine/cloperastine treatment.

FIG. 28 depicts the normalized effect of breathing for control, morphine and cloperastine measured against morphine for Te and Ti duration and breath frequency and ventilation.

FIG. 29 depicts the effects on firing activity of rostral ventral respiratory group (rVRG) neurons in a control animal.

FIG. 30 depicts the effects on firing activity of rostral ventral respiratory group (rVRG) neurons in an animal given 50 mg/kg of morphine.

FIG. 31 depicts the return to near normal firing activity of rostral ventral respiratory group (rVRG) neurons in an animal given 50 mg/kg of morphine followed by 30 mg/kg off cloperastine.

FIG. 32 is a graph which compares the firing duration in milliseconds of a subject animal given 50 mg/kg of morphine, and animal receiving 50 mg/kg of morphine and 30 mg/kg off cloperastine versus control.

FIG. 33 is a graph which compares the breathing frequency in Hz of a subject animal given 50 mg/kg of morphine, and animal receiving 50 mg/kg of morphine and 30 mg/kg off cloperastine versus control.

FIG. 34 is a graph which compares the number of spikes/breath of a subject animal given 50 mg/kg of morphine, and animal receiving 50 mg/kg of morphine and 30 mg/kg off cloperastine versus control.

FIG. 35 is a graph which compares the firing duration in milliseconds of a subject animal given 30 mg/kg off cloperastine, an animal receiving 50 mg/kg of morphine and 30 mg/kg of cloperastine versus control.

FIG. 36 is a graph which compares the breathing frequency in Hz of a subject animal given 30 mg/kg off cloperastine, an animal receiving 50 mg/kg of morphine and 30 mg/kg of cloperastine versus control.

FIG. 37 is a graph which compares the number of spikes /breath of a subject animal given 30 mg/kg off cloperastine, an animal receiving 50 mg/kg of morphine and 30 mg/kg of cloperastine versus control.

FIG. 38 depicts the normalized effect on rVRG for control, morphine and cloperastine measured against morphine for firing duration, frequency and spikes/breath.

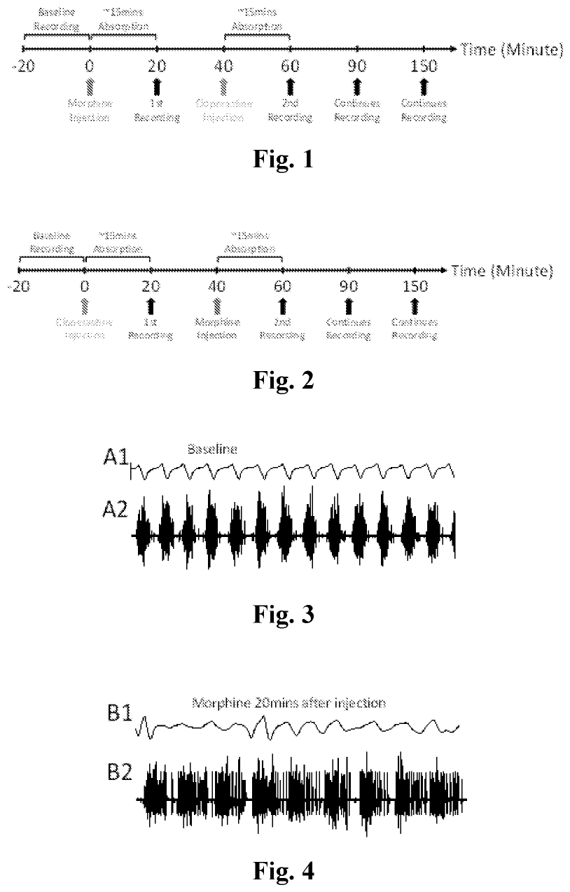

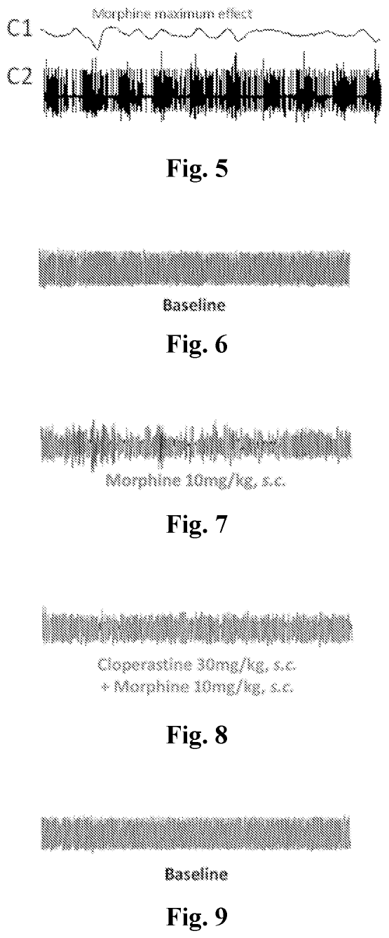

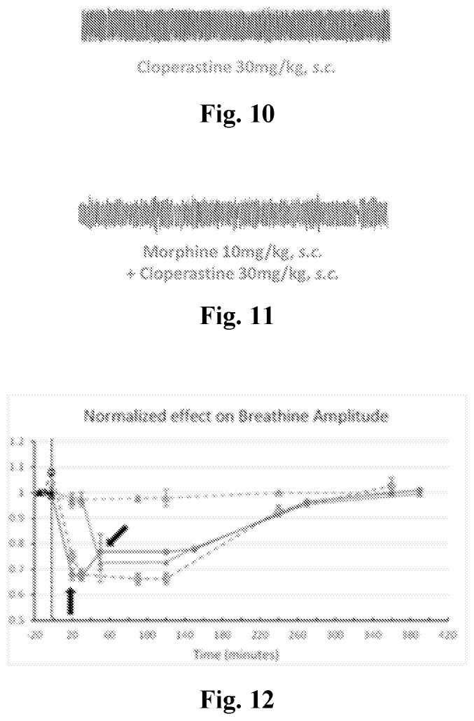

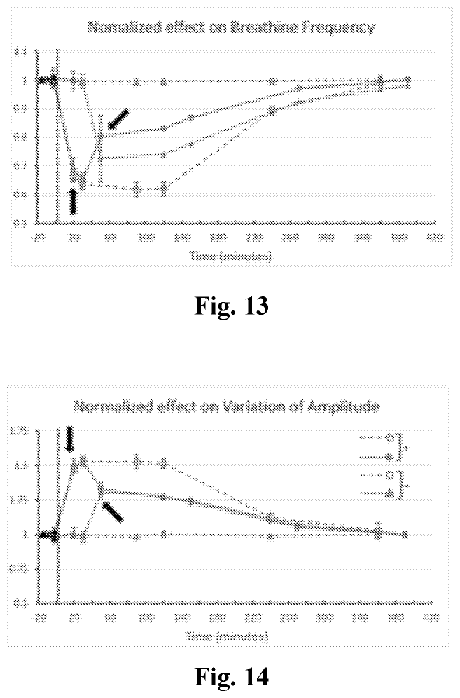

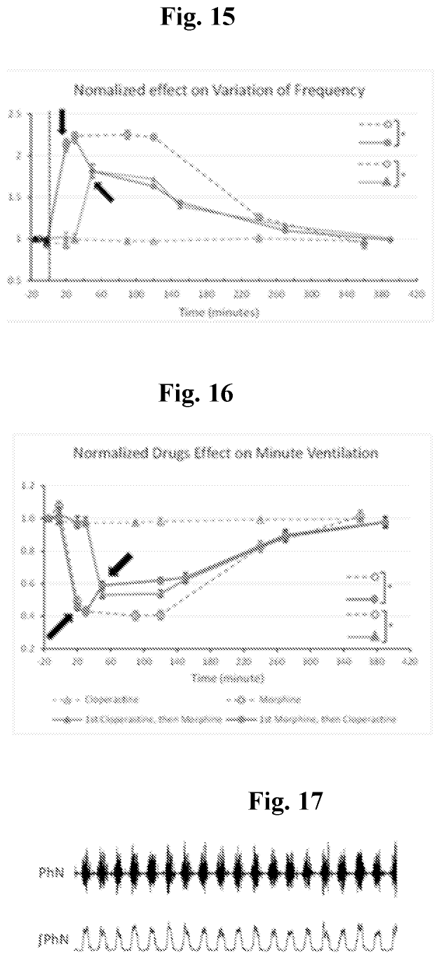

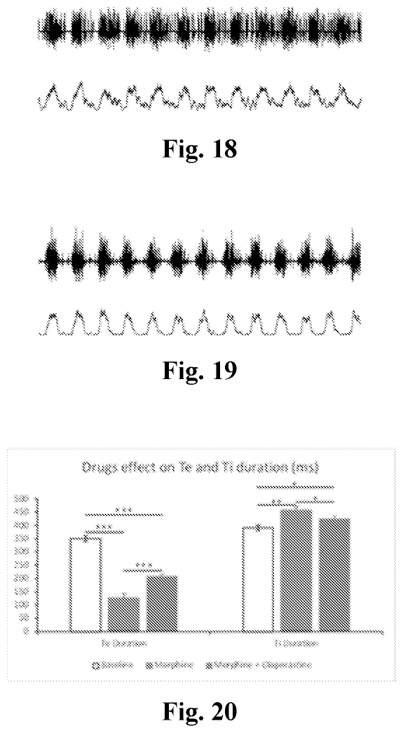

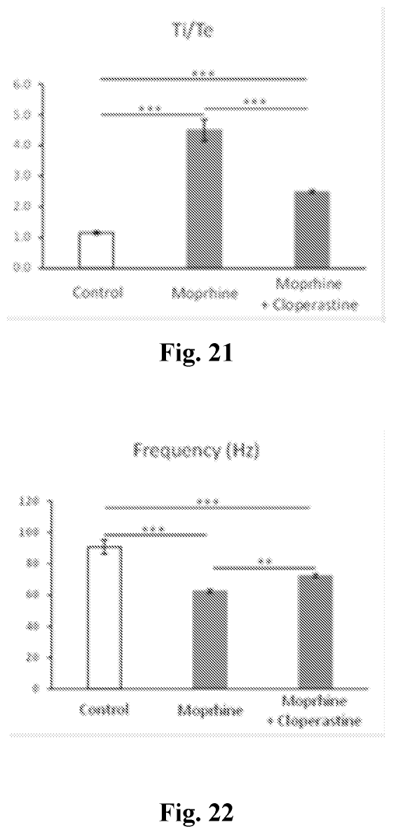

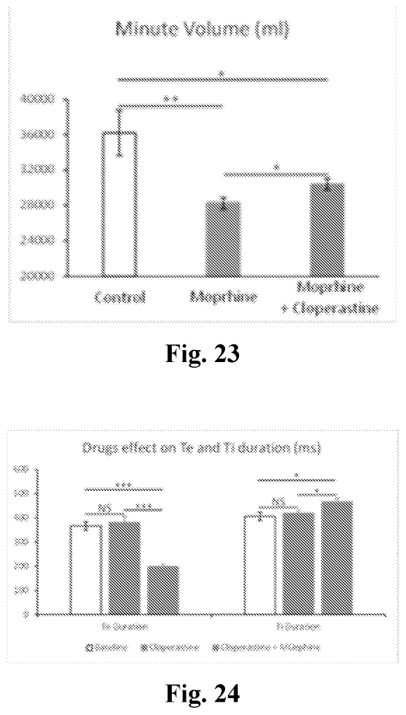

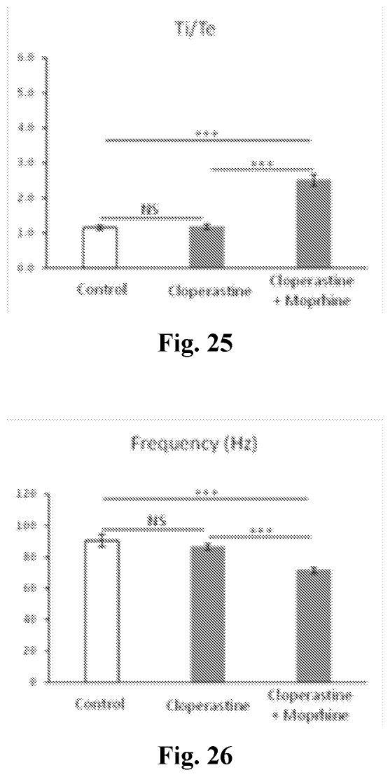

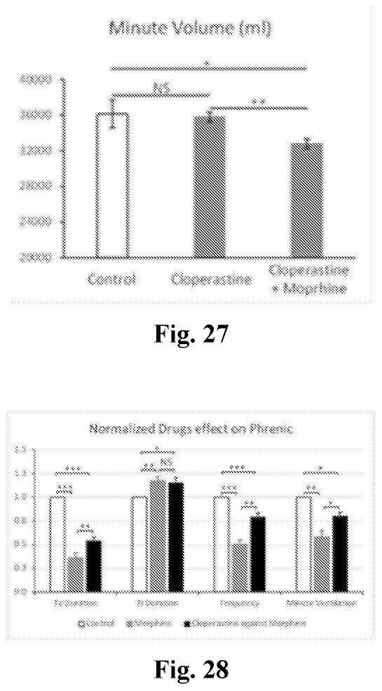

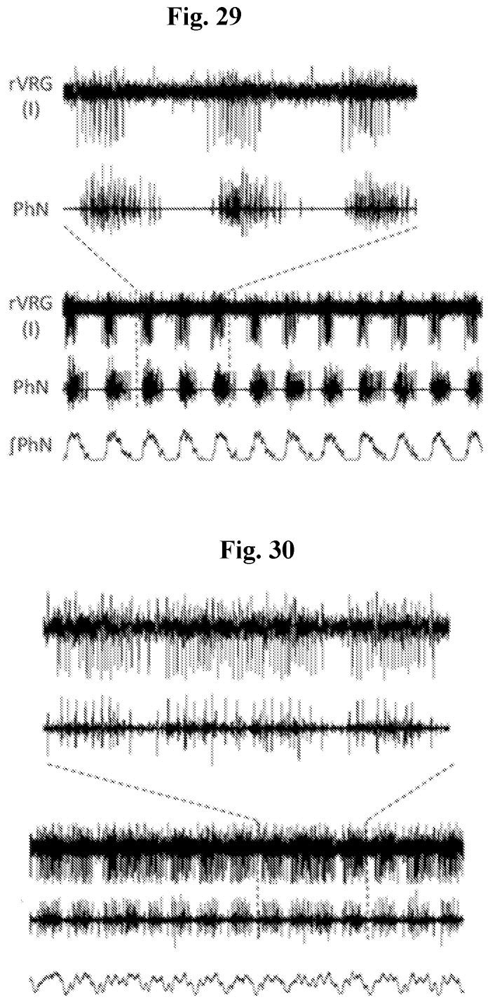

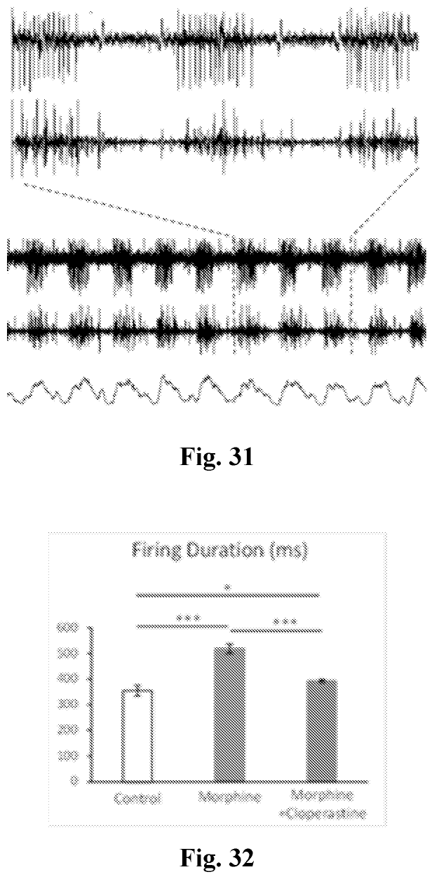

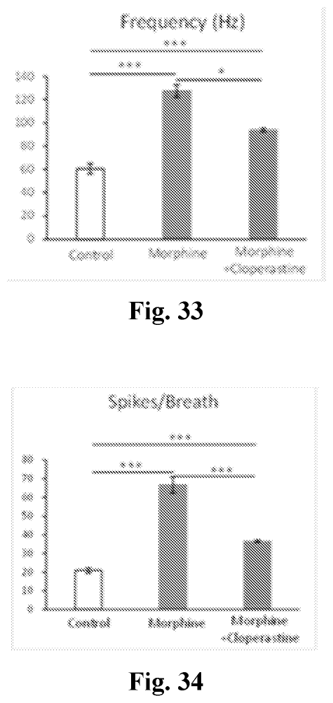

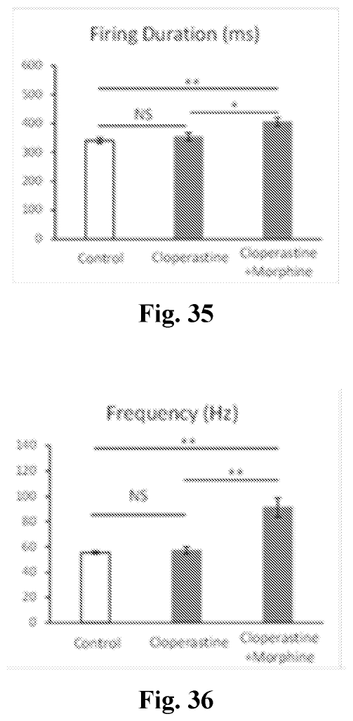

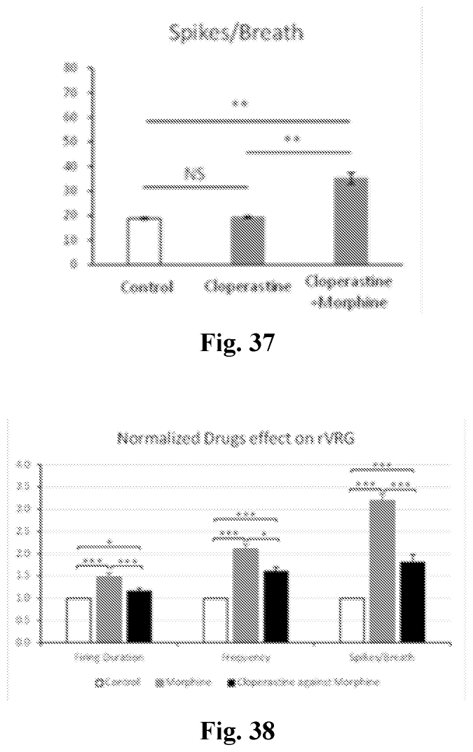

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

The terms “a” and “an” are defined as one or more unless this disclosure explicitly requires otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance can or cannot occur, and that the description includes instances where the event or circumstance occurs and instances where it does not.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Any embodiment of any of the compounds and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Disclosed is a method for reducing morphine-induced breathing, phrenic and rVRG abnormal activities utilizing the antitussive and antihistamine cloperastine. It has been established that 10 mg/kg morphine causes central hypoventilation with decreased breathing minute ventilation, increased variation of both breathing amplitude & frequency.

A major adverse effect of the chronic use of opioid drugs is respiratory depression causing a loss of a large number in the United States. Via μ-Opioid receptors (MOR), respiratory neurons in the brainstem are known to be the targets, especially those in the ventral respiratory column (VRC). However, how the VRC neurons react to chronic opioid drugs remains elusive. To understand how chronic morphine exposures affect brainstem respiratory motor output, we performed these studies in Sprague Dawley rats, using repetitive morphine administrations. Consistent with previous reports, severe breathing depression was found in these rats. Since the MOR is coupled to GIRK channels, we applied Cloperastine, a GIRK channel blocker and a commercial OTC drug, to the rats before or after chronic morphine. Cloperastine reverses hypoventilation mainly by suppression the irregularity of breathing amplitude and frequency. These results suggest that the respiratory depression after chronic morphine exposures does not seem due to general suppression of brainstem respiratory motor output solely, while corruptions in rhythmic regulation of the motor output may play a more important role involving the GIRK channel as its inhibitor Cloperastine was capable of counteracting chronic morphine-induced central hypoventilation.

Disclosed herein are methods for treating chronic morphine exposures and how this exposure affects brainstem respiratory motor output. Disclosed herein are studies using Sprague Dawley rats, during which repetitive morphine administrations were conducted. Following the chronic treatments, the same dose morphine suppressed the minute ventilation by 55.2%±4.1 (n=16 rats) for 3-4 hours accompanied with severe variations in tidal volume and breathing frequency as measured in plethysmography. These effects reached a plateau level in 3-4 days. Recording from these rats in spontaneous breathing after decerebration, we disclose the findings of similar changes in breathing activity in phrenic discharges.

Ectopic phrenic activity during expiration was seen in these rats after chronic morphine exposures. Firing activity of respiratory neurons was recorded extracellularly in the ventral respiratory group. A large number of E-I phase-spanning neurons were observed in rats treated with chronic morphine but not with saline injection. These results suggest that the respiratory depression after chronic morphine exposures does not seem due to general suppression of brain-stem respiratory motor output solely, while corruptions in rhythmic regulation of the motor output may play a more important role.

### Methods

Disclosed herein are methods for modulating the effects of an opioid in a subject, comprising administering to a subject an effective amount of cloperastine to reduce the effects of the opioid on breathing. Non-limiting examples of the effects of opioids relates to phrenic and rVRG abnormal activities.

In one embodiment the amount of cloperastine is effective in minimizing the effects of an opioid. What is meant by minimizing in one embodiment is to return breathing, phrenic and rVRG abnormal activities to 50% of normal values. In another embodiment it is to return breathing, phrenic and rVRG abnormal activities to 70% of normal values. In a further embodiment it means to return breathing, phrenic and rVRG abnormal activities to 75% of normal values. In a yet further embodiment it means to return breathing, phrenic and rVRG abnormal activities to 80% of normal values. In a still further embodiment it means to return breathing, phrenic and rVRG abnormal activities to 90% of normal values.

In a yet still further embodiment it means to return breathing, phrenic and rVRG abnormal activities back to normal values.

The amount of cloperastine administered can be from about 1 mg/kg to about 100 mg/kg for every 100 mg/kg of an opioid. The following are non-limiting amounts of cloperastine that can be administered for every 100 mg/kg of an opioid in a subject 1 mg/kg, 2 mg/kg, 3 mg/kg, 4 mg/kg, 5 mg/kg, 6 mg/kg, 7 mg/kg, 8 mg/kg, 9 mg/kg 10 mg/kg, 11 mg/kg, 12 m, 13 mg/kg, 14 mg/kg, 15 mg/kg, 16 mg/kg, 17 mg/kg, 18 mg/kg, 19 mg/kg, 20 mg/kg, 21 mg/kg, 22 m, 23 mg/kg, 24 mg/kg, 25 mg/kg, 26 mg/kg, 27 mg/kg, 28 mg/kg, 29 mg/kg, 30 mg/kg, 31 mg/kg, 32 m, 33 mg/kg, 34 mg/kg, 35 mg/kg, 36 mg/kg, 37 mg/kg, 38 mg/kg, 39 mg/kg, 40 mg/kg, 41 mg/kg, 42 m, 43 mg/kg, 44 mg/kg, 45 mg/kg, 46 mg/kg, 47 mg/kg, 48 mg/kg, 49 mg/kg, 50 mg/kg, 51 mg/kg, 52 m, 53 mg/kg, 54 mg/kg, 55 mg/kg, 56 mg/kg, 57 mg/kg, 58 mg/kg, 59 mg/kg, 60 mg/kg, 61 mg/kg, 62 m, 63 mg/kg, 64 mg/kg, 65 mg/kg, 66 mg/kg, 67 mg/kg, 68 mg/kg, 69 mg/kg, 70 mg/kg, 71 mg/kg, 72 m, 73 mg/kg, 74 mg/kg, 75 mg/kg, 76 mg/kg, 77 mg/kg, 78 mg/kg, 79 mg/kg, 80 mg/kg, 81 mg/kg, 82 m, 83 mg/kg, 84 mg/kg, 85 mg/kg, 86 mg/kg, 87 mg/kg, 88 mg/kg, 89 mg/kg, 90 mg/kg, 91 mg/kg, 92 m, 93 mg/kg, 94 mg/kg, 95 mg/kg, 96 mg/kg, 97 mg/kg, 98 mg/kg, 99 mg/kg, or 100 mg/kg.

The amount of cloperastine administered can be adjusted depending upon the amount of opioid in the subjects system. The following are non-limiting amounts of an opioid that can be present in a subjects system 1 mg/kg, 2 mg/kg, 3 mg/kg, 4 mg/kg, 5 mg/kg, 6 mg/kg, 7 mg/kg, 8 mg/kg, 9 mg/kg 10 mg/kg, 11 mg/kg, 12 m, 13 mg/kg, 14 mg/kg, 15 mg/kg, 16 mg/kg, 17 mg/kg, 18 mg/kg, 19 mg/kg, 20 mg/kg, 21 mg/kg, 22 m, 23 mg/kg, 24 mg/kg, 25 mg/kg, 26 mg/kg, 27 mg/kg, 28 mg/kg, 29 mg/kg, 30 mg/kg, 31 mg/kg, 32 m, 33 mg/kg, 34 mg/kg, 35 mg/kg, 36 mg/kg, 37 mg/kg, 38 mg/kg, 39 mg/kg, 40 mg/kg, 41 mg/kg, 42 m, 43 mg/kg, 44 mg/kg, 45 mg/kg, 46 mg/kg, 47 mg/kg, 48 mg/kg, 49 mg/kg, 50 mg/kg, 51 mg/kg, 52 m, 53 mg/kg, 54 mg/kg, 55 mg/kg, 56 mg/kg, 57 mg/kg, 58 mg/kg, 59 mg/kg, 60 mg/kg, 61 mg/kg, 62 m, 63 mg/kg, 64 mg/kg, 65 mg/kg, 66 mg/kg, 67 mg/kg, 68 mg/kg, 69 mg/kg, 70 mg/kg, 71 mg/kg, 72 m, 73 mg/kg, 74 mg/kg, 75 mg/kg, 76 mg/kg, 77 mg/kg, 78 mg/kg, 79 mg/kg, 80 mg/kg, 81 mg/kg, 82 m, 83 mg/kg, 84 mg/kg, 85 mg/kg, 86 mg/kg, 87 mg/kg, 88 mg/kg, 89 mg/kg, 90 mg/kg, 91 mg/kg, 92 m, 93 mg/kg, 94 mg/kg, 95 mg/kg, 96 mg/kg, 97 mg/kg, 98 mg/kg, 99 mg/kg, or100 mg/kg.

In another embodiment the ratio of cloperastine to the opioid can be from about 1 mg/kg to about 1 mg/kg to about 100 mg/kg1 mg/kg:1 mg/kg. Non-limiting examples include: 1 mg/kg:1 mg/kg, 1 mg/kg:2 mg/kg, 1 mg/kg:3 mg/kg, 1 mg/kg:4 mg/kg, 1 mg/kg:5 mg/kg, 1 mg/kg:6 mg/kg, 1 mg/kg:7 mg/kg, 1 mg/kg:8 mg/kg, 1 mg/kg:9 mg/kg, 1 mg/kg:10 mg/, 1, 1 mg/kg:11 mg/, 1 mg/kg:12 mg/kg, 1 mg/kg:13 mg/kg, 1 mg/kg:14 mg/kg, 1 mg/kg:15 mg/kg, 1 mg/kg:16 mg/kg, 1 mg/kg:17 mg/kg, 1 mg/kg:18 mg/kg, 1 mg/kg:19 mg/kg, 1 mg/kg:20 mg/kg, 1 mg/kg:21 mg/kg, 1 mg/kg:22 ml mg/kg:23 mg/kg, 1 mg/kg:24 mg/kg, 1 mg/kg:25 mg/kg, 1 mg/kg:26 mg/kg, 1 mg/kg:27 mg/kg, 1 mg/kg:28 mg/kg, 1 mg/kg:29 mg/kg, 1 mg/kg:30 mg/kg, 1 mg/kg:31 mg/kg, 1 mg/kg:32 ml mg/kg:33 mg/kg, 1 mg/kg:34 mg/kg, 1 mg/kg:35 mg/kg, 1 mg/kg:36 mg/kg, 1 mg/kg:37 mg/kg, 1 mg/kg:38 mg/kg, 1 mg/kg:39 mg/kg, 1 mg/kg:40 mg/kg, 1 mg/kg:41 mg/kg, 1 mg/kg:42 ml mg/kg:43 mg/kg, 1 mg/kg:44 mg/kg, 1 mg/kg:45 mg/kg, 1 mg/kg:46 mg/kg, 1 mg/kg:47 mg/kg, 1 mg/kg:48 mg/kg, 1 mg/kg:49 mg/kg, 1 mg/kg:50 mg/kg, 1 mg/kg:51 mg/kg, 1 mg/kg:52 ml mg/kg:53 mg/kg, 1 mg/kg:54 mg/kg, 1 mg/kg:55 mg/kg, 1 mg/kg:56 mg/kg, 1 mg/kg:57 mg/kg, 1 mg/kg:58 mg/kg, 1 mg/kg:59 mg/kg, 1 mg/kg:60 mg/kg, 1 mg/kg:61 mg/kg, 1 mg/kg:62 ml mg/kg:63 mg/kg, 1 mg/kg:64 mg/kg, 1 mg/kg:65 mg/kg, 1 mg/kg:66 mg/kg, 1 mg/kg:67 mg/kg, 1 mg/kg:68 mg/kg, 1 mg/kg:69 mg/kg, 1 mg/kg:70 mg/kg, 1 mg/kg:71 mg/kg, 1 mg/kg:72 ml mg/kg:73 mg/kg, 1 mg/kg:74 mg/kg, 1 mg/kg:75 mg/kg, 1 mg/kg:76 mg/kg, 1 mg/kg:77 mg/kg, 1 mg/kg:78 mg/kg, 1 mg/kg:79 mg/kg, 1 mg/kg:80 mg/kg, 1 mg/kg:81 mg/kg, 1 mg/kg:82 ml mg/kg:83 mg/kg, 1 mg/kg:84 mg/kg, 1 mg/kg:85 mg/kg, 1 mg/kg:86 mg/kg, 1 mg/kg:87 mg/kg, 1 mg/kg:88 mg/kg, 1 mg/kg:89 mg/kg, 1 mg/kg:90 mg/kg, 1 mg/kg:91 mg/kg, 1 mg/kg:92 m 1 mg/kg:93 mg/kg, 1 mg/kg:94 mg/kg, 1 mg/kg:95 mg/kg, 1 mg/kg:96 mg/kg, 1 mg/kg:97 mg/kg, 1 mg/kg:98 mg/kg, 1 mg/kg:99 mg/kg, or 1 mg/kg:100 mg/kg.

In a still further embodiment the ratio of the opioid to cloperastine can be from about 1 mg/kg to about 1 mg/kg to about 100 mg/kg. Non-limiting examples include: 1 mg/kg:1 mg/kg, 1 mg/kg:2 mg/kg, 1 mg/kg:3 mg/kg, 1 mg/kg:4 mg/kg, 1 mg/kg:5 mg/kg, 1 mg/kg:6 mg/kg, 1 mg/kg:7 mg/kg, 1 mg/kg:8 mg/kg, 1 mg/kg:9 mg/kg, 1 mg/kg:10 mg/, 1, 1 mg/kg:11 mg/, 1 mg/kg:12 mg/kg, 1 mg/kg:13 mg/kg, 1 mg/kg:14 mg/kg, 1 mg/kg:15 mg/kg, 1 mg/kg:16 mg/kg, 1 mg/kg:17 mg/kg, 1 mg/kg:18 mg/kg, 1 mg/kg:19 mg/kg, 1 mg/kg:20 mg/kg, 1 mg/kg:21 mg/kg, 1 mg/kg:22 ml mg/kg:23 mg/kg, 1 mg/kg:24 mg/kg, 1 mg/kg:25 mg/kg, 1 mg/kg:26 mg/kg, 1 mg/kg:27 mg/kg, 1 mg/kg:28 mg/kg, 1 mg/kg:29 mg/kg, 1 mg/kg:30 mg/kg, 1 mg/kg:31 mg/kg, 1 mg/kg:32 ml mg/kg:33 mg/kg, 1 mg/kg:34 mg/kg, 1 mg/kg:35 mg/kg, 1 mg/kg:36 mg/kg, 1 mg/kg:37 mg/kg, 1 mg/kg:38 mg/kg, 1 mg/kg:39 mg/kg, 1 mg/kg:40 mg/kg, 1 mg/kg:41 mg/kg, 1 mg/kg:42 ml mg/kg:43 mg/kg, 1 mg/kg:44 mg/kg, 1 mg/kg:45 mg/kg, 1 mg/kg:46 mg/kg, 1 mg/kg:47 mg/kg, 1 mg/kg:48 mg/kg, 1 mg/kg:49 mg/kg, 1 mg/kg:50 mg/kg, 1 mg/kg:51 mg/kg, 1 mg/kg:52 ml mg/kg:53 mg/kg, 1 mg/kg:54 mg/kg, 1 mg/kg:55 mg/kg, 1 mg/kg:56 mg/kg, 1 mg/kg:57 mg/kg, 1 mg/kg:58 mg/kg, 1 mg/kg:59 mg/kg, 1 mg/kg:60 mg/kg, 1 mg/kg:61 mg/kg, 1 mg/kg:62 ml mg/kg:63 mg/kg, 1 mg/kg:64 mg/kg, 1 mg/kg:65 mg/kg, 1 mg/kg:66 mg/kg, 1 mg/kg:67 mg/kg, 1 mg/kg:68 mg/kg, 1 mg/kg:69 mg/kg, 1 mg/kg:70 mg/kg, 1 mg/kg:71 mg/kg, 1 mg/kg:72 m 1 mg/kg:73 mg/kg, 1 mg/kg:74 mg/kg, 1 mg/kg:75 mg/kg, 1 mg/kg:76 mg/kg, 1 mg/kg:77 mg/kg, 1 mg/kg:78 mg/kg, 1 mg/kg:79 mg/kg, 1 mg/kg:80 mg/kg, 1 mg/kg:81 mg/kg, 1 mg/kg:82 ml mg/kg:83 mg/kg, 1 mg/kg:84 mg/kg, 1 mg/kg:85 mg/kg, 1 mg/kg:86 mg/kg, 1 mg/kg:87 mg/kg, 1 mg/kg:88 mg/kg, 1 mg/kg:89 mg/kg, 1 mg/kg:90 mg/kg, 1 mg/kg:91 mg/kg, 1 mg/kg:92 ml mg/kg:93 mg/kg, 1 mg/kg:94 mg/kg, 1 mg/kg:95 mg/kg, 1 mg/kg:96 mg/kg, 1 mg/kg:97 mg/kg, 1 mg/kg:98 mg/kg, 1 mg/kg:99 mg/kg, or 1 mg/kg:100 mg/kg.

Further disclosed is a method for modulating the effects of an opioid in a subject, comprising administering to a subject an effective amount of cloperastine to reduce the effects of the opioid on breathing, phrenic and rVRG abnormal activities wherein the desirable effects of the opioid are not reduced. A non-limiting desirable effect of an opioid is analgesia.

1. A method for modulating the effects of an opioid in a subject, comprising administering to a subject an effective amount of cloperastine to reduce the effects of the opioid on breathing.

2. The method according to claim 1, wherein the opioid affects the μ-receptor.

3. The method according to claim 1, wherein the opioid is morphine.

4. The method according to claim 1, wherein the opioid affects the δ-receptor.

5. The method according to claim 1, wherein the opioid affects the κ-receptor.

6. The method according to claim 1, wherein breathing abnormalities are reduced.

7. The method according to claim 6, wherein the breathing abnormality is hypoventilation.

8. The method according to claim 1, wherein any irregular respiratory rhythm and inspiratory-expiratory patterns are reduced.

9. The method according to claim 1, wherein any adverse effects on the phrenic nerve is reduced.

10. The method according to claim 1, wherein the amount of cloperastine is from about 1 mg/kg to about 100 mg/kg for every 100 mg/kg of an opioid.

11. The method according to claim 1, wherein the amount of the opioid is from about 1 mg/kg to about 100 mg/kg for every 100 mg/kg of cloperastine.

12. The method according to claim 1, wherein the ratio of cloperastine to the opioid can be from about 1 mg/kg to about 1 mg/kg to about 100 mg/kg 1 mg/kg:1 mg/kg.

13. The method according to claim 1, wherein the ratio of the opioid to cloperastine can be from about 1 mg/kg to about 1 mg/kg to about 100 mg/kg

14. The method according to claim 1, wherein the amount of cloperastine administered to the subject is at least about 30 mg/kg for every 10 mg/kg in the opioid.

15. The method according to claim 1, wherein the beneficial effects of the opioid is not reduced.

16. The method according to claim 15, wherein the beneficial effect is analgesia.

17. A method for prophylactically modulating the effects of an opioid in a subject, comprising administering to a subject an effective amount of cloperastine to reduce the effects of an opioid which is suspected to be taken by a subject on breathing.

18. The method according to claim 17, wherein the opioid affects the μ-receptor.

19. The method according to claim 17, wherein the opioid is morphine.

20. The method according to claim 17, wherein the opioid affects the δ-receptor.

21. The method according to claim 17, wherein the opioid affects the κ-receptor.

22. The method according to claim 17, wherein breathing abnormalities are reduced.

23. The method according to claim 22, wherein the breathing abnormality is hypoventilation.

24. The method according to claim 17, wherein any irregular respiratory rhythm and inspiratory-expiratory patterns are reduced.

25. The method according to claim 17, wherein any adverse effects on the phrenic nerve is reduced.

26. The method according to claim 17, wherein the amount of the opioid is from about 1 mg/kg to about 100 mg/kg for every 100 mg/kg of cloperastine.

27. The method according to claim 17, wherein the ratio of cloperastine to the opioid can be from about 1 mg/kg to about 1 mg/kg to about 100 mg/kg 1 mg/kg:1 mg/kg.

28. The method according to claim 17, wherein the ratio of the opioid to cloperastine can be from about 1 mg/kg to about 1 mg/kg to about 100 mg/kg

29. The method according to claim 17, wherein the amount of cloperastine administered to the subject is at least about 30 mg/kg for every 10 mg/kg of the opioid that the subject is suspected to take.
